(19) United States
(12) Patent Application Publication
(10) Pub. No.: US 2005/0177215 A1
(43) Pub. Date: Aug. 11, 2005

(54) Method and device for lower extremity cryo-thermo therapy

(76) Inventor: Steven L. Rosenberg, Santa Monica, CA (US)

Correspondence Address: PERKINS COIE LLP, POST OFFICE BOX 1208, SEATTLE, WA, 98111-1208, US

(21) Appl. No.: 11/055,927

(22) Filed: Feb. 11, 2005

Related U.S. Application Data
(60) Provisional application No. 60/543,512, filed on Feb. 11, 2004.

(51) Int. Cl. A61F 7/00

(52) U.S. Cl. 607/111

(57) ABSTRACT

Disclosed is a method and device for cryotherapy and thermotherapy using a cryo-thermotherapeutic device. The device comprises an upper and lower material or an inner and outer material depending on the anatomy treated. The device further comprises a substance within the upper and lower or inner and outer materials that can be repeatedly heated and cooled. Separate designs of the device allow the cryo-thermotherapeutic device to be used in the lower or upper extremity. Further, the cryo-thermotherapeutic device may comprise a means for measuring temperature that is located within the substance or as a part of the upper, lower, inner or outer material.

### PRIORITY CLAIM

The present application claims priority to U.S. Provisional Application Ser. No. 60/543,512, filed Feb. 11, 2004, which is incorporated herein by reference.

### FIELD OF THE INVENTION

The present invention is related to the field of physiotherapy for the lower extremity, particularly the feet.

### BACKGROUND OF THE INVENTION

Breakthroughs in physiotherapy have enhanced athletic performance, weight training, general well being and activities of dialing living. In many cases the physiotherapy is directed to a particular body part, muscle group or joint using heat or cold. The goal has been to increase movement, affect healing of an injured area, increase or decrease blood flow, reduce edema, “cool down” from athletic exertion and to relax, to name a few. Many of the modalities have been directed to the lower extremity and particularly the feet.

Pain, swelling or stiffness in the foot disturbs body equilibrium, feeling of wellness, energy and proprioception and often causes symptoms in the hip, knee, spine and neck. By elevating and massaging the foot, more proximal areas of the anatomy also respond relieving many symptoms, including anxiety. Further, significant literature has been devoted to the so called “visceral zones” in the soles of the feet in an effort to explain the amount of relief that can be gained in proximal body areas by massaging or otherwise treating the feet.

This treatment, often called reflexology of the feet divides the foot into zones designated to represent other parts of the body. The soles of the feet are particularly associated with the viscera, which are said to respond when the corresponding zone in the foot is massaged or manipulated. Regardless of the complete credence of reflexology, a number of people swear by its relaxing effects. Further, other modalities have been recognized as ancient forms of this therapeutic concept including foot acupuncture, cautery and venesection. THE FOOT, (Helal, et al., Ed., Churchill Livingstone, 1988). Better known modalities include ultrasound, joint mobilization, muscle strengthening, balance training, ice, heat, whirlpools, interferential therapy, pulsed magnetic fields, transcutaneous nerve stimulation (TNS), biofeedback, pharmacotherapy, and psychology.

Treatment modalities are affected by a given person's aesthesia, biomechanics, vascular supply and venous drainage, lymphatics, weight, bone strength, age and general health. The local physiology of the area is of utmost importance. The foot is a load bearing and propulsion system created by a specific balance of forces between articular surfaces, soft tissues and both intrinsic and extrinsic musculature. Very few modalities address each of these structures. However, two of the most inexpensive and readily available modalities, heat and cold, can be used for each of these structures.

Heat in the form of warm water, wax, paraphyn, or infrared radiation to name a few, is commonly used. Although heat is often thought of as an all encompassing physical therapy modality, in reality, heat on its own is not of great benefit in the foot. Further, heat may exacerbate reduced venous tone when applied to the foot. Patients with neuropathy, incompetent veins and foot edema have generally been advised to avoid heat treatments, especially those that apply heat to the foot. Using current heat modalities, it is difficult to avoid the greater and lesser saphenous veins of the foot, thereby decreasing venous tone.

Due to the drawbacks with heat therapy, therapies have been developed which use heat in combination with cold. Most commonly, a patient is advised to soak the selected anatomy in hot water for a prescribed time and then switch to cold water. Compliance is often problematic with this type of treatment due to the time involved, necessity of appropriate baths, water temperature and mess associated with soaking. A device is needed which can focus the application of heat to an area of the foot without creating many of the drawbacks common to heat treatment.

At least one method of cold therapy is ice broken into small fragments mixed with cold water wherein a patient soaks the affected area. Cold packs and bulky wraps for the cold pack are also used on the foot. While the modality is simple, compliance may be low due to the bulky dressing involved and time necessary to fix a cold pack to a selected area.

Most hot and cold therapies require a patient to remain in one area, seated, for the best effect of the treatment. A treatment modality is needed which allows specific areas to be treated without being bulky or calling for wrapping or other binding of the modality to the selected anatomy. Further, a treatment modality is needed that allows a patient to be mobile. Additional difficulties exist for those patients with any type of peripheral neuropathy. Often, a patient will suffer burns or excessive numbing and limited blood flow secondary to cold exposure. A device is needed that easily allows appropriate temperature ranges without relying on sensation to adjust for the appropriate temperature.

### SUMMARY OF THE INVENTION

The invention is directed to a device and method for a cryo-thermo therapy. In one embodiment of the invention the cryo-thermotherapeutic device is comprised of an upper material and a lower material in the shape of the plantar aspect of the foot including a medial arch extension. The upper and lower materials are fastened together to create a hollow center or cavity. The cavity is filled with a substance capable of repeated cooling and heating.

In another embodiment of the invention a cryo-thermotherapeutic device is comprised of an upper material and a lower material in the shape of the plantar aspect of the foot including a medial arch extension. The upper and lower materials are fastened together to create a plurality of hollow centers or cavities. Preferably, an anterior cavity is separate from a posterior cavity. The plurality of cavities are filled with a substance capable of repeated cooling and heating.

Another embodiment of the invention a cryo-thermotherapeutic device is comprised of an upper and lower material in the shape of the plantar aspect of the foot including a medial arch extension. The upper and lower materials are fastened or fixed together to create a hollow center or cavity. The cavity is filled with a substance capable of repeated cooling and heating. The cryo-thermotherapeutic device further comprises a means for measuring the temperature of the substance. Another aspect of a similar embodiment the cryo-thermotherapeutic device comprises a plurality of cavities. The plurality of cavities may be separated from each other to accommodate different foot sizes and shapes. It is contemplated that the cryo-thermotherapeutic device will be available in men's and woman's small, medium and large and that further sizing can be accomplished by trimming out cavities from the cryo-thermotherapeutic device.

In another embodiment of the invention a cryo-thermotherapeutic device is comprised of an upper material and a lower material wherein the upper material is of a different durometer than the lower material and wherein the upper material and lower material are in the shape of the plantar aspect of the foot including a medial arch extension. The upper and lower materials are fastened together to create a single or a multiple of hollow centers or cavities. The cavity or cavities are filled with a substance capable of repeated heating and cooling.

In another embodiment, a first cryo-thermotherapeutic device comprises an upper material and a lower material shaped in the form of a plantar foot wherein the upper material and the lower material are fixed to each other in such a way to create at least one cavity between the fixed upper and lower material, and wherein the cavity is filled with a substance capable of capable of being heated or cooled, and including a second cryo-thermotherapeutic device that comprises an upper and a lower material in the form of a dorsal foot wherein the upper material and the lower material are fixed to each other in such a way to create at least one cavity and wherein the cavity is filled with a substance capable of being heated or cooled. The first and second cryo-thermotherapeutic devices are elastically fixed to each other to accommodate a foot within a space between the first and second cryo-thermotherapeutic devices. In another embodiment of the invention a cryo-thermotherapeutic device as described above is shaped for use at the proximal foot, ankle and leg.

In another embodiment of the invention a cryo-thermotherapeutic device has differing types of material for the upper and lower material. Further, the upper and lower materials, whether the same or differing, may be of differing thickness.

In another embodiment of the invention the cryo-thermotherapeutic device(s) described above is used in a method of cryotherapy or thermotherapy or a combination thereof for treatment of foot, ankle and leg ailments or for palliative measures.

### BRIEF DESCRIPTION OF THE FIGURES

FIG. 1 illustrates an anatomical chart showing the 4 plantar muscular layers of the foot.

FIG. 2 illustrates a superior view of a plantar cryo-thermotherapeutic device.

FIG. 3 illustrates a lateral view of a plantar cryo-thermotherapeutic device.

### DETAILED DESCRIPTION OF THE PREFERRED EMBODIMENTS

All references cited above and below are incorporated by reference as if fully set forth herein.

The instant invention is primarily directed to a cryo-thermotherapeutic device (CTTD) for the treatment of pathology of the foot and for palliative relief and comfort. While it is contemplated that the instant invention can be designed for and used in more proximal areas of the lower extremity, and that the instant invention could be designed for and used in the hand and arm, preferred treatments using the invention are focused on specific deep and surface anatomy of the foot and ankle as well as visceral zones of the foot.

The plantar, or lower, aspect of the foot is commonly divided into four facial layers. The amount of heat and cold and time of application will be calculated upon the layer to be treated. Put in simpler terms, the colder and longer applications will be used for deeper structures in the foot. Similarly, with heat, warmer and longer applications of heat are used for deeper structures of the foot. For more complicated pathology of the foot and ankle or leg, a podiatrist or other practitioner skilled in the art will know the appropriate use for the CTTD based on the structure(s) involved.

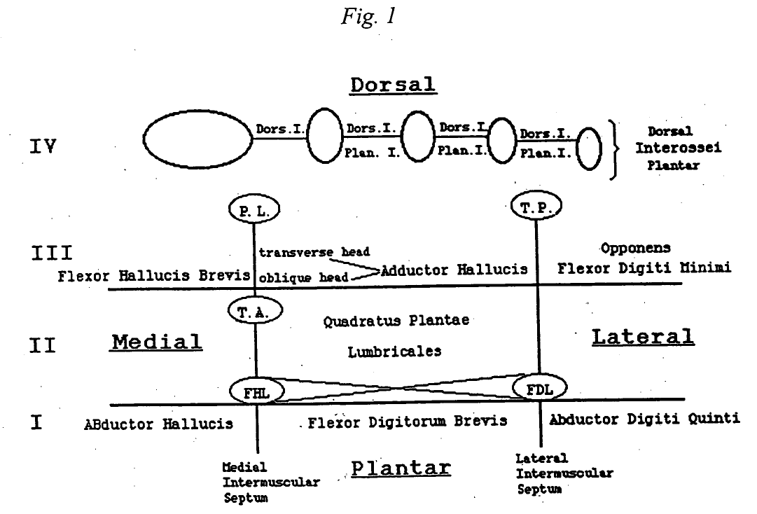

FIG. 1 illustrates how anatomists commonly divide the plantar foot musculature in to four layers. The first or most planer layer contains the abductor hallucis, the flexor digitorum brevis, the abductor digiti minimi (also called the abductor digiti quinti). The three plantar muscles are separated by two plantar to dorsal intermuscular septa called the medial intermuscular septum and the lateral intermuscular septum and are supplied by the medial and lateral plantar nerves. Specifically, the flexor digitorum brevis is covered superficially by the deep surface of the plantar aponeurosis. This relationship is important to the treatment of many disorders associated with plantar fascitis and plantar calcaneal exostosis, or “heel spur.”

The second plantar layer, moving dorsally, contains the tendons of the flexor hallucis longus and the flexor digitorum longus. In between the medial and lateral intermuscular septa reside the quadratus plantae and four lumbricale muscules. The muscles of the second layer are supplied by the medial and lateral plantar nerves.

The muscles contained in the third layer of the foot are the flexor hallucis brevis, adductor hallucis and flexor digiti minimi brevis. These muscles are supplied by the proper digital branch of the medial plantar nerve, deep branch of the lateral plantar nerve and the superficial branch of the lateral plantar nerve.

The fourth layer of muscles is contained primarily between the lesser metatarsals of the foot and contain the three plantar interossei and the four dorsal interossei. The deep and superficial branches of the lateral plantar nerve supply the fourth layer muscles.

Each of the four plantar facial compartments is interwoven with smaller nerves, the medial and lateral plantar arteries and branches and the associated venous networks. Further, two critical structures are plantar to the first plantar facial compartment; the panniculosus adiposus, or plantar fat pad, and the plantar aponeurosis, or plantar fascia. These structures are more separately divided posteriorly and more intertwined anteriorly. See, Fundamentals of Foot Surgery, McGlamery, Ed. (Williams and Wilkins, 1987).

The skin, superficial fascia and deep fascia are supplied by branches of the sural, medial plantar cutaneous, lateral plantar cutaneous, lateral dorsal cutaneous, intermediate dorsal cutaneous, and the terminal branch of the deep peroneal nerve. Due to the complex relationship of the nerves and communicating branches, inflammations, injuries or any sensation producing stimuli may be referred to nervous “networks” of the foot, ankle and leg. The current invention works to treat the primarily affected areas as well as areas where pain or other sensation is being referred.

The CTTD is designed to heat or cool the plantar surface of the foot, or, in separate embodiments, the whole foot, ankle and leg. They are primarily therapeutic for foot pain and also used as a foot comfort product when the feet get erythematous. The CTTD designed for the plantar aspect of the foot treats foot problems such as heel pain, plantar fascitis, bursitis, capsulitis, metatarsalgia, muscle spasms, muscle strains and any other pain related to the lower anatomy of the foot that can be effectively treated with a therapeutic heat and/or ice modality. The CTTD is designed for use in a person's shoes, slippers, socks, sandals or other footgear. The design allows a person to use the CTTD with ease and avoids unwanted taping, bandages or bias to hold the hot/cold therapy to the foot or other affected area.

It one embodiment of the invention a CTTD is shaped to the contours of the plantar foot in normal anatomy. A portion of the CTTD is designed to fill the medial arch of the foot using the foot gear to mold the CCDT to the medial arch as well as other areas. The CTTD is filled with a substance suitable for multiple treatments involving heat or cold therapy. The CTTD can be preformed or trimed to fit men or women's shoes, socks, slippers or other footgear. The CTTD is preferably gel filled and is temperature sensitive to heat and cold and will freeze when placed in the freezer. The CTTD is preferably made of two different thickness of an upper and lower polyethylene material. Preferably, the upper material is made of 8 mils thick blue polyethylene and the lower material is 12 mils thick of clear polyethylene. Differing thickness may be used in each material and are appropriate in a range of at least about 5-16 mm.

The CTTD is made by cutting the polyethylene material into two rectangles, one for the upper material and one for the lower material. The clear lower material is placed on a vacuum mold. The lower material is heated, then vacuumed molded to a shape that creates two separate open cavities that will nest a gel. The first open cavity runs from the posterior most aspect of the rear foot to the metatarsophalangeal joint area of the foot and a second open cavity that runs from the metatarsophalangeal joint area of the foot to the distal most aspect of the forefoot. The depths of the open cavities are approximately 1/4″ deep to the superficial upper material. A gel is then placed in the open cavities. The upper material of blue polyethylene is placed over the clear polyethylene lower material and a radio frequency or other sealing machine is used to seal the upper and lower polyethylene materials together. The product is then die cut to the actual plantar aspect foot shape.

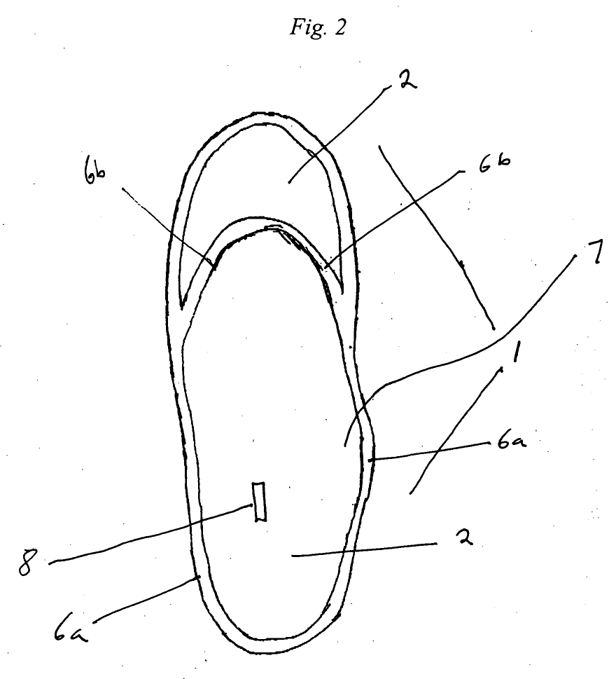

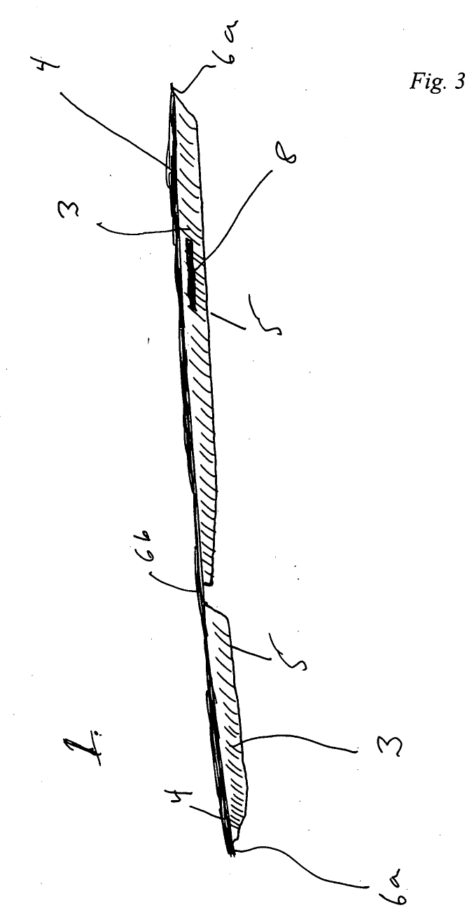

The cut of the CTTD is in the shape of the rectus or slightly pronated plantar foot having a forefoot, mid-foot and rear foot anatomical shape in the transverse plane and includes a medial extension at the arch. FIG. 2 illustrates a superior view of the CTTD 1 and FIG. 3 illustrates a lateral view of the CTTD 1. In FIG. 3 it can be seen that there is a cavity 2 in the CTTD capable of nesting a substance 3 capable of multiple heating or cooling.

In the slightly pronated arrangement, the CTTD 1 occupies a more flat arrangement than the normally arched foot. The CTTD 1 contains an upper material 4 and a lower material 5 that are fastened together 6a, 6b so as to create the above mentioned plurality of cavities 2 which can be filled with a substance 3 which can be heated and cooled repeatedly. The multiple cavities may be separated from each other to accommodate different foot sizes and shapes. It is contemplated that the cryo-thermotherapeutic device will be available in men's and woman's small, medium and large and that further sizing can be accomplished by trimming out cavities from the cryo-thermotherapeutic device. For example, in FIG. 2, the CTTD may be trimmed or cut or separated at 6b to accommodate a smaller foot. FIG. 2 further illustrates a medial arch extension 7. In this respect the CTTD 1 facilitates formation to the plantar aspect of the foot.

The upper material 4 and lower material 5 should be of such a strength that multiple heating and cooling do not deform or weaken the material. Further, the upper material 4 and lower material 5 should be strong enough, yet flexible enough to allow minimum to moderate weight bearing. While it is contemplated that full weight bearing be a part of an appropriate use for certain embodiments of the CTTD 1, it is preferred that it be constructed in such a way to create flexibility over rigidity.

Upper and Lower materials for the CTTD may be selected from a group that consists of plastics metals or composites. Some examples include polyurethane polyethylene, polypropylene, rubber, neoprene, silicone, and water resistant materials such as Gortex® and the like. Moreover, it is contemplated that the upper material 4 and the lower material 5 can be different material and/or of different durometer.

In another embodiment of the invention, a means for measuring temperature 8 is combined either within the substance 3 in the cavity 2 or affixed to the upper or lower material 4,5.

The means for measuring the temperature may be a strip type thermometer well known in the medical arts or the substance in the cavity may contain a chemical with known reversible thermochromic characteristics when differing temperatures are applied.

Reversible thermochromic compositions are known which are constituted of a solubilized mixture containing essentially a reaction medium for causing reversible electron exchange between an electron-donating coloring organic compound and an electron-accepting organic compound in a specific temperature range as disclosed in U.S. Pat. Nos. 4,028,118; 4,732,810; 4,865,648; 4,720,301; 4,957,949; 4,554,565; 4,421,560; 4,425,161; and 4,421,560.

Additional examples of reversible thermochromic materials that can be used in the present invention include those disclosed in U.S. Pat. No. 5,281,570 (Hasegawa, et al.) entitled “Thermochromic materials” teaching a microencapsulated reversible thermochromic material comprising: (a) an electron donative color former; (b) a sulfide, sulfoxide or sulfone containing a hydroxy phenyl radical; and (c) a chemical compound selected from alcohols, esters, ethers, ketones, carboxylic acids or acid amides; U.S. Pat. No. 4,425,161 (Shibahashi, et al.) entitled “Thermochromic materials” teaching a thermochromic material comprising (a) an electron-donating, chromatic organic compound, (b) a compound capable of reversibly accepting an electron or electrons from the electron-donating, chromatic organic compound, (c) a compound controlling the temperature and sensitivity of coloration/decoloration of the thermochromic material and (d) a N-radical, P-radical, O-radical or S-radical cationic compound having an aromatic ring or rings which improves by light-fastness; U.S. Pat. No. 4,717,710 (Shimizu, et al.) entitled “Thermochromic composition” teaching a thermochromic composition comprising (1) an electron-donating chromogenic material, (2) a 1,2,3-triazole compound, (3) a weakly basic, sparingly soluble azomethine or carboxylic acid primary amine salt, and (4) an alcohol, amide or ester serving as a solvent; and U.S. Pat. No. 5,558,700 (Shibahashi, et al.) entitled “Reversible thermochromic composition” teaching a reversible thermochromic composition comprising a solubilized mixture of three components of (a) an electron-donating color-developing organic compound selected from pyridine types, quinazoline types, and bisquinazoline types of compound, (b) an electron-accepting compound for the electron-donating color-developing organic compound, and (c) a compound serving as a reaction medium for causing a reversible electron exchange reaction between the components (a) and (b) within a specified temperature range, the composition developing a fluorescent color of yellow, yellowish orange, orange, reddish orange, or red with a high color density and high color brightness, yet gives no residual color under non-color-developing conditions, and has remarkably improved light resistance.

A preferred thermochromic composition used is one that is chromogenic throughout a temperature range of 30 degrees to 180 degrees Fahrenheit, which is the range of skin freezing to skin burning, respectively. Thermochromic compositions within this range are taught in U.S. Pat. No. 6,290,977 (Friars, et al).

In another embodiment of the invention the CTTD is filled with a substance that is a solid. The solid substance may be a single solid substance or a plurality of solid substances. Preferably this plurality of solid substances are beads, 2 to 4 mm in diameter and composed of plastic, metal, ceramic or composites. Examples include polymers, methyl methacrylate or light weight metal beads. Another example is a bead that is solid but deformable such as rubber. In this embodiment of the invention, the wearer of the CTTD may use the CTTD during ambulation.

In another embodiment of the invention, a CTTD is designed for use to extend proximally up the foot, ankle or leg. Thus, the device is preformed to fit the normal anatomy of the foot ankle or leg and a patient may slip on a CTTD device that is made up of two or more CTTD devices that are elastically attached to each other. The elastic attachments may be anything from a sock to separate elastic straps well known in the art. For example, a first cryo-thermotherapeutic device and a second cryo-thermotherapeutic device may be attached with elastic straps wherein the first cryo-thermotherapeutic device is in the shape of a plantar foot and the second cryo-thermotherapeutic device is in the shape of a dorsal foot. The elastically attached CTTDs may be placed over the foot (or other affected area) and held it in place. The CTTD of this embodiment is formed using the same process as described above wherein the vacuum mold comports with the anatomical design to be achieved. In the designs for more proximal use it is necessary to create separate cavities to hold the substance for heat and cold and prevent gravitational shift of certain chosen substances such as gel. Therefore, the same fixing mechanism to bring the upper and lower materials together will be used to create a series of cavities within the CTTD to hold portions of the substance for heat and cold. In this respect, the upper and lower materials are more correctly referred to an inner and outer material. The CTTD for more proximal use may also include the means for measuring temperature described above.

In another embodiment of the invention, the CTTD is designed for the hand and arm and is and worn as a glove.

The foregoing descriptions of embodiments of the invention are intended to be illustrative of the embodiments of at least one aspect of the invention, and are not intended to limit the invention in any way. Although the invention has been described with respect to specific modifications, the details thereof are not to be construed as limitations, for it will be apparent that various equivalents, changes and modifications may be resorted to without departing from the spirit and scope thereof, and it is understood that such equivalent embodiments are to be included herein. At least one aspect of the invention is further illustrated by the following examples, which are not intended to limit the effective scope of the claims.

### EXAMPLE 1

An active 23-year-old male presents to a doctor's office with a chief complaint of sweating and hot feet after exercise. The heat and sweating are out of proportion to the exercise. The doctor determines that there is no pathological reason for the diaphoresis and erythema. The doctor then prescribes a gel-filled cryo-thermotherapeutic device as described above. The use of the CTTD is ordered for 20 to 30 minutes after exercise with the temperature of the CTTD between 40-45 degrees Fahrenheit. Upon the patient's next visit, the patient describes relief of his symptoms. The doctor then informs the patient that bead-filled CTTDs are available and may be used during the patient's exercise.

### EXAMPLE 2

An active 43-year-old female runner presents to the doctor's office with a chief complaint of pain in the ball of the right foot. She states the pain is worse while running, but is constant throughout the day and sometimes at night. The doctor determines that the pain is secondary to an overuse syndrome causing metatarsalgia and capsulitis. The doctor informs the patient that she will need a cortisone injection and some oral anti-inflammatory agents. The patient tells the doctor that she is not interested in an injection and never takes medicine that is not natural. The doctor then prescribes for the patent a gel-filled CTTD as described above. The patient is to use the CTTDs in a contrast fashion wherein a cold CTTD is used for 2 minutes and a heat CTTD is used for 6 minutes. The patient is ordered to repeat the hot/cold applications of the CTTDs for 24 minutes at least four times a day. The patient is advised that contrast therapy may be accomplished without the use of the CTTDs by placing the foot in separate baths of cold and hot water. The patient elects for the CTTD as she feels her compliance is likely to be much higher with the avoidance of the mess associated with soaking and the convenience of the CTTD. Upon the patient's return the doctor's office, she admits to an 85 percent relief with the use of contrast CTTDs.

### EXAMPLE 3

A 50 year-old female housewife has aching feet nearly every day due to housework and activities of daily living. She has been warned against applying heat to her feet as she has a history of peripheral neuropathy secondary to insulin dependent diabetes mellitus. She goes to the drug store to seek relief that does not include heat soaks; pads and the like. While at the store she notices the cryo-thermotherapeutic device under the trade name COOL SOLES® and notices the device has the ability to measure temperature. She then consults with her doctor over the telephone about this new product. The doctor informs her of the safe temperature ranges and approves her use of the cryo-thermotherapeutic device.

After reading the description and uses for the device, she purchases an over-the-counter version of the gel-filled device that contains a means for measuring temperature and uses it at home. She uses the CTTD as directed by her doctor and the package insert and gains relief from use of the CTTD.

1. A cryo-thermotherapeutic device comprising an upper material and a lower material shaped in the form of a plantar foot and wherein the upper material and the lower material are fixed to each other in such a way to create at least one cavity between the fixed upper and lower material and wherein the cavity is filled with a substance capable of being heating and cooled.

2. The device of claim 1 wherein the upper material and the lower material are composed of a type of material selected from the group of the group consisting of plastic, metal or composites and combinations thereof.

3. The device of claim 2 wherein the plastic is polyethylene.

4. The device of claim 1 wherein the substance is a liquid or a solid.

5. The device of claim 4 wherein the liquid is a gel.

6. The device of claim 5 wherein the gel is a water based gel.

7. The device of claim 4 wherein the substance is a plurality of solid beads.

8. The device of claim 7 wherein the solid beads are composed of a plastic, metal, ceramic or composite.

9. The device of claim 7 wherein the solid beads are 0 to 5 mm in diameter.

10. The device of claim 9 wherein the beads are 1.5-2.5 mm in diameter.

11. The device of claim 1 further comprising a means for measuring temperature of the substance.

12. The device of claim 11 wherein the means for measuring temperature is located within the substance.

13. The device of claim 1 wherein the upper material and the lower material are different types of materials.

14. The device of claim 13 wherein the upper material and the lower material are of different thickness.

15. A cryo-thermotherapeutic device comprising an upper material and a lower material shaped in the form of a plantar foot wherein the upper and lower material are fixed to each other in such a way to create at least one cavity wherein the cavities are filled with a substance capable of being heated or cooled and wherein the device further comprises a means for measuring temperature.

16. The device of claim 15 wherein the means for measuring temperature is located within the substance.

17. The device of claim 15 wherein the means for measuring temperature is located on the upper material or the lower material.

18. The device of claim 15 wherein the upper material and the lower material are different types of materials.

19. The device of claim 18 wherein the upper material and the lower material are of different thickness.

20. A first cryo-thermotherapeutic device comprising an upper material and a lower material shaped in the form of a plantar foot wherein the upper material and the lower material are fixed to each other in such a way to create at least one cavity between the fixed upper and lower material, and wherein the cavity is filled with a substance capable of capable of being heated or cooled, and a second cryo-thermotherapeutic device comprising an upper and a lower material in the form of a dorsal foot wherein the upper material and the lower material are fixed to each other in such a way to create at least one cavity and wherein the cavity is filled with a substance capable of being heated or cooled, and wherein the first and second cryo-thermotherapeutic devices are elastically attached to each other to accommodate a foot within a space between the first and second cryo-thermotherapeutic devices.

21. The first and second cryo-thermotherapeutic devices of claim 20 wherein one or both the first and second cryo-thermotherapeutic devices further comprises a means for measuring temperature.
